(19) United States
(12) Patent Application Publication
(10) Pub. No.: US 2006/0292654 A1
(43) Pub. Date: Dec. 28, 2006

(54) MICROWELL PLATE

(75) Inventor: Paul Reardon, Weybridge (GB)

Correspondence Address: AKERMAN SENTERFITT, P.O. BOX 3188, WEST PALM BEACH, FL, 33402-3188, US

(73) Assignee: INOGEN, S.A., Gland (CH)

(21) Appl. No.: 11/425,611

(22) Filed: Jun. 21, 2006

(30) Foreign Application Priority Data
Jun. 28, 2005 (GB) 0513086.9

(51) Int. Cl. C12Q 1/48

(52) U.S. Cl. 435/15

(57) ABSTRACT

The shallower wells of a crystalisation plate in which it is intended to form crystals are part-spherical and of uniform wall thickness. The optical effect of the transparent plastics material from which the plate is moulded counter the distortion of a view through a microscope so that crystals viewed through the microscope appear to be on a flat surface. Associated with the wells are markings so small that they can be read through the microscope, making it unnecessary for the user to take the eye off the microscope to determine the position on the plate.

This Application is related to and claims priority from British Patent Application No. GB 0513086,9 filed Jun. 28, 2005.

This invention relates to an improved microwell plate of the kind used for crystallisation, cell culture and general biological research applications.

### BACKGROUND OF THE INVENTION

Such microwell plates are used for the investigative screening of biological samples. A plate has multiple formations each comprising a relatively deeper well and at least one relatively shallower well formed in a common depression in the top surface of the plate. A biological sample is placed in the shallower well and in the deeper well there is placed a solution of chemicals designed to promote the growth of crystals in the shallower well by vapour deposition. As the solution evaporates in some cases a crystal will form in the shallower well or wells from the vapour. The shallower wells are examined under a microscope and when a crystal is found it is removed for analysis by pipette.

A principal object of the present invention is to provide an improved microwell plate.

### SUMMARY OF THE INVENTION

In accordance with the invention there is provided a microwell plate for crystalisation, cell culture and general biological research applications comprising multiple formations each comprising a deeper well and at least one transparent, relatively shallower well in an upper surface of the plate, wherein the shallower well is of curved cross section to be upwardly concave and generally uniform wall thickness.

Preferably the shallower well is part-spherical. The radius of curvature of the sphere is preferably calculated equally with respect to its top and bottom surface to compensate for the refractive index of the plastics polymer used to manufacture the plate.

The wells of each formation are preferably within a common depression. The depth of said depression in the vicinity of the shallower well or wells is preferably in the region of not more than 800 microns.

Two similar relatively shallower wells may be associated with each relatively deeper well.

Preferably the formations are arranged in columns with lands separating the columns so that adhesive tape can be applied across parallel lands on each side of a column to seal the formations therein.

The plate is preferably moulded from a transparent plastics material.

Individual indicia of dimensions such as to be viewed through a microscope may be marked on the plate adjacent the shallower wells. The indicia are preferably letters and/or numerals having a height in the region of 0.8 mm and a width in the region of 0.1 mm.

### BRIEF DESCRIPTION OF THE DRAWINGS

A preferred embodiment of the invention will now be described by way of non-limitative example with reference to the accompanying drawings, in which:

FIG. 1 is a plan view of a microwell plate in accordance with the invention,

FIG. 2 is a sectional elevation taken on the line II-II of FIG. 1,

FIG. 3 is a view on an enlarged scale of one of the formations 3 of the plate of FIGS. 1 and 2, and

FIG. 4 is a sectional elevation taken on the line IV-IV of FIG. 3.

### DETAILED DESCRIPTION OF A PREFERRED EMBODIMENT

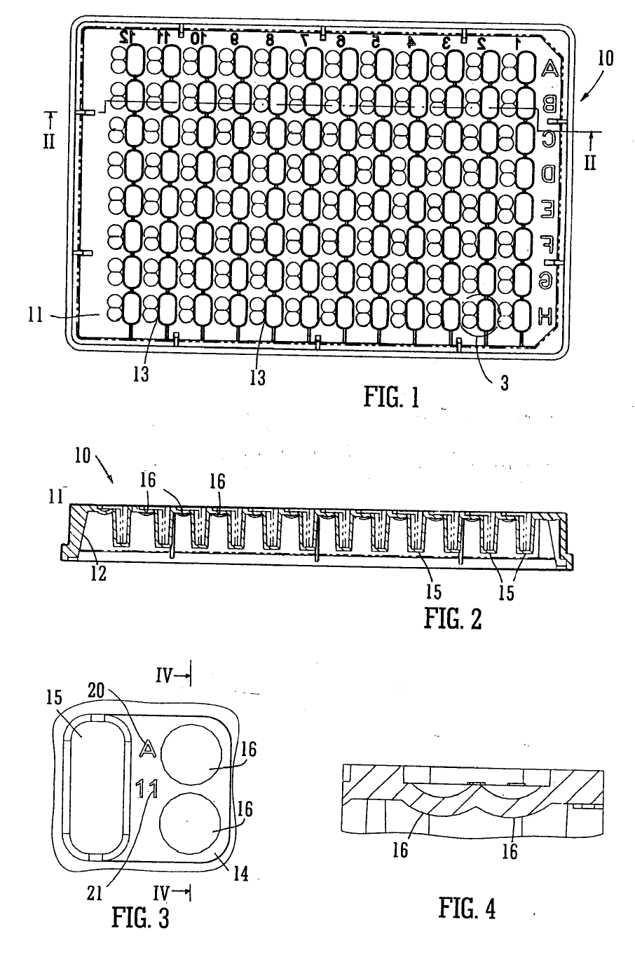

The microwell plate 10 of FIGS. 1 and 2 is moulded from a transparent plastics material to have a flat upper surface 11 supported by a peripheral skirt 12. In the upper surface 11 multiple formations 3 are formed in columns separated by parallel lands 13. Each formation 3 comprises a very shallow, generally rectangular depression 14 within which a relatively deep well 15 is formed as well as two shallow wells 16. As is most clear from FIG. 4 each shallow well 16 is of curved cross section to be upwardly concave with a uniform wall thickness. When a specimen in a shallow well 16 is inspected under a microscope it appears flat, the curved appearance normal when viewing through a microscope having been countered by the optical properties of the plastics material. This is a surprising discovery. The “obvious” solution would be to form the well 16 as a concave lens. Instead it is found that the downwardly convex shape illustrated best serves this purpose and compensates for the refractive index of the plastics polymer used in the construction of the plate.

In a preferred embodiment of the invention each depression is not more than 800 microns in depth and the radius of curvature of each part-spherical shallow well 16 is in the region of 5 degrees.

With these dimensions it is relatively easy to pipette crystals out of the shallow wells 16. The lands 13 between the columns permit the wells of all formations 3 of a common column to be sealed by a an adhesive tape (not shown) applied to parallel lands.

In accordance with another feature of the present invention unique indicia, such as letters and numerals 20,21 (FIG. 4) are marked in the depression 14 of each formation between the deeper well 15 and the shallower wells 16. Each indices is so small as to be almost invisible to the naked eye but can be read easily through a microscope. This means that an operator can identify the particular formation 3 he is looking at without having to take his eye from the microscope. This greatly accelerates the examination process with consequent saving of the operator's time. Preferably the indicia 20,21 are each no more than 0.8 mm high and no more than 1 mm wide.

I claim:

1. A microwell plate for crystalisation, cell culture and general biological research applications comprising multiple formations each comprising a deeper well and at least one transparent, relatively shallower well in an upper surface of the plate, wherein the shallower well is of curved cross section to be upwardly concave and of generally uniform wall thickness.

2. A plate as claimed in claim 1, wherein the shallower well is part-spherical.

3. A plate as claimed in claim 2, wherein the radius of curvature of the sphere is in the region of 5 degrees.

4. A plate as claimed in claim 1, wherein the wells of each formation are within a common depression in the upper surface of the plate.

5. A plate as claimed in claim 4, wherein the depth of each said depression in the vicinity of the shallower well or wells is in the region of not more than 800 microns.

6. A plate as claimed in claim 1, wherein two similar relatively shallower wells are associated with each relatively deeper well.

7. A plate as claimed in claim 1, wherein the formations are arranged in columns with lands separating the columns so that adhesive tape can be applied across parallel lands on each side of a column to seal the formations therein.

8. A plate as claimed in claim 1 moulded from a transparent plastics material.

9. A plate as claimed in claim 1, wherein individual indicia of dimensions such as to be viewed through a microscope are marked on the plate adjacent the shallower wells.

10. A plate as claimed in claim 9 wherein the indicia are letters and/or numerals having a height in the region of 0.8 mm and a width in the region of 0.1 mm.
